(19) United States
(12) Patent Application Publication
(10) Pub. No.: US 2016/0151055 A1
(43) Pub. Date: Jun. 2, 2016

(54) BIOPSY DEVICE AND METHOD FOR OBTAINING A TOMOGRAM OF A TISSUE VOLUME USING SAME

(71) Applicants: THE ROYAL INSTITUTION FOR THE ADVACEMENT OF LEARNING/MCGILL UNIVERSITY, Montréal (CA); UNIVERSITY HEALTH NETWORK, Toronto (CA); POLYVALOR, LIMITED PARTNERSHIP, Montreal (CA)

(72) Inventors: Frederic LEBLOND, Outremont (CA); Brian WILSON, Toronto (CA); Andreanne GOYETTE, Montreal (CA); Julien PICHETTE, Montreal (CA); Kevin PETRECCA, Verdun (CA); Caroline BOUDOUX, Montreal (CA); Wendy-Julie MADORE, Montreal (CA); Mathias STRUPLER, Montreal (CA)

(21) Appl. No.: 14/906,065

(22) PCT Filed: Jul. 25, 2014
(86) PCT No.: PCT/CA2014/050708

§ 371 Date: Jan. 19, 2016

Related U.S. Application Data
(60) Provisional application No. 61/858,867, filed on Jul. 26, 2013.

(51) Int. Cl. A61B 10/04; A61B 10/02; A61B 5/1455; A61B 5/145; A61B 1/07; A61B 5/1459

(52) U.S. Cl. CPC A61B 10/04; A61B 1/07; A61B 5/1459; A61B 5/14556; A61B 5/14546; A61B 10/0275; A61B 2010/045

(57) ABSTRACT

The biopsy device generally comprises: a cannula body having a longitudinal axis and a probing region extending along the longitudinal axis, the probing region having a sample receiving window defined therein for receiving a sample of a surrounding tissue when performing a biopsy; and a plurality of optical fibers mounted along an exterior portion of the cannula body, each of the plurality of optical fibers having a fiber end in the probing region of the cannula body, at least one of the plurality of optical fibers being adapted to illuminate the surrounding tissue with an optical signal generated by the at least one light generator and at least one of the plurality of optical fibers being adapted to detect an optical signal response with the at least one light detector, the optical signal response being caused by the propagation of the optical signal in the surrounding tissue.

### REFERENCE TO RELATED APPLICATIONS

This patent application claims priority of U.S. provisional Application Ser. No. 61/858,867, filed on Jul. 26, 2013, the contents of which are hereby incorporated by reference.

### FIELD OF THE APPLICATION

The present application relates to biopsy needles of the type used for stereotactic needle biopsies, such as brain needle biopsies.

### BACKGROUND OF THE ART

Brain tumors represent a non-negligible portion of all new cancer cases. The standard-of-care is open-cranium surgery to achieve maximum resection, which markedly affects survival, followed by radiation and chemotherapy. The decision to resect is informed by the suspected type and grade of the tumor, which is roughly evaluated by pre-operative magnetic resonance imaging (hereinafter, MRI) or computed tomography (hereinafter, CT), and by the risk/benefit assessment based on the likelihood of permanent neurological deficit for lesions located in eloquent cortex or other critical structures.

When precise neuropathological diagnosis is required, a stereotactic brain needle biopsy (hereinafter, BNB) is performed to collect tissue prior to or in place of surgery, providing information (tumor type, grade, growth pattern) that is more sensitive and specific than imaging. The main indications for BNB are deep-seated lesions, multiple lesions and lesions in surgical candidates with poor health. The BNB procedure begins with a determination of where and approximately how many specimens can be acquired based on the preoperative images. These images are spatially co-registered with the needle in the operating room, allowing the needle to be positioned in the targeted areas using a neuronavigation system (e.g., Stealth Station from the company Medtronic). Typically, numerous needle passes (serial biopsies) can be required to ensure that the most relevant pathological information is available to determine optimal treatment.

In conventional BNBs, a biopsy needle comprises internal and external cannulas, a needle stop and an aspirator tube. The external cannula (approximately 2 mm diameter) is affixed to a rigid stereotactic frame and the needle is slowly inserted through a burr hole in the skull. Both cannulas have an open rectangular window into which tissue is drawn by suction, at which point the internal cannula is rotated to cut the specimen, and then withdrawn to secure the sample.

The primary goal in BNB is to sample the most malignant part of the lesion. If the procedure is successful then the correct diagnosis is made by histopathological analysis and no further steps are required. However, the current standard-of-care can lead to misdiagnosis because of geographic miss due to registration inaccuracies or patient movement secondary to the registration process. Inadequate tumor specificity in the preoperative images used to guide biopsy may also contribute to misdiagnosis. In practice, an important portion of BNB cases require a second procedure because non-diagnostic samples are taken in, for example, necrotic or normal tissue. Moreover, some procedures yield non-representative samples due to inappropriate definition of the target, which leads to incorrect tumor typing and grading. To reduce misdiagnosis, intraoperative neuropathological assessment of frozen sections is sometimes performed during the procedure, but this is time consuming, costly and significantly less definitive and accurate than histopathology on stained sections, which takes several days.

A further significant limitation is intracranial hemorrhage, the rate of which varies widely and which may contribute to morbidity and mortality. This is caused by rupturing a significant blood vessel that is aspirated into the inner cannula during cutting. The surgeon does not have access to the location of blood vessels during BNB; hence, this risk is always present. Thus, despite being much less invasive than open-cranium surgery, BNB carries significant risks.

### SUMMARY OF THE APPLICATION

There is provided a biopsy device that addresses issues related to the prior art. There is provided a biopsy device incorporating a plurality of optical fibers disposed on an exterior surface thereof for illuminating with an optical signal a surrounding tissue volume and for detecting an optical signal response caused by the propagation of the optical signal in the surrounding tissue volume. The optical signal response may provide information to reduce the risk of misdiagnosis and hemorrhages. The biopsy devices of the present disclosure may be used to detect and quantify tumor-specific endogenous or exogenous optical biomarkers locally or in a tomographic manner using an optical image reconstruction algorithm. The biopsy device may be used in a spectroscopic biopsy system for providing guidance to a mechanical biopsy procedure which may include multiple detection schemes such as fluorescence detection and/or reflectance detection and/or inelastic scattering (e.g. Raman spectroscopy signals).

Therefore, in accordance with a first embodiment, there is provided a biopsy device comprising: a cannula body having a longitudinal axis and a probing region extending along the longitudinal axis, the probing region having a sample receiving window defined therein for receiving a sample of a surrounding tissue when performing a biopsy; and a plurality of optical fibers mounted along an exterior portion of the cannula body, each of the plurality of optical fibers having a fiber end in the probing region of the cannula body and another fiber end optically connectable to at least one of at least one light generator and at least one light detector, at least one of the plurality of optical fibers being adapted to illuminate the surrounding tissue with an optical signal generated by the at least one light generator and at least one of the plurality of optical fibers being adapted to detect an optical signal response with the at least one light detector, the optical signal response being caused by the propagation of the optical signal in the surrounding tissue.

Further in accordance with the first embodiment, the plurality of optical fibers are circumferentially spaced from one another.

Still further in accordance with the first embodiment, each one of the fiber ends of the plurality of optical fibers are circumferentially aligned with one another.

Still further in accordance with the first embodiment, each one of the fiber ends is optically coupled to a portion of an annular redirecting surface being abutted with the circumferentially aligned fiber ends of the plurality of optical fibers, the annular redirecting surface having a normal axis forming a non-perpendicular and non-zero angle with the longitudinal axis.

Still further in accordance with the first embodiment, the annular redirecting surface is a polished cylindrical tube.

Still further in accordance with the first embodiment, each one of the fiber ends is optically coupled to a redirecting surface being aligned along an angled axis forming a non-zero angle with the longitudinal axis of the cannula body.

Still further in accordance with the first embodiment, the redirecting surface is deposited on a prism abutted to a corresponding fiber end.

Still further in accordance with the first embodiment, each one of the fiber ends of the plurality of optical fibers longitudinally extends to the sample receiving window.

Still further in accordance with the first embodiment, the plurality optical fibers are circumferentially and evenly distributed on the exterior portion of the cannula body.

Still further in accordance with the first embodiment, each one of the fiber ends of the plurality of optical fibers extends at least on a circumferential side of the cannula body which is opposite to the sample receiving window.

Still further in accordance with the first embodiment, at least one optical fiber of the plurality of optical fibers is adapted to illuminate the surrounding tissue with an optical signal and to detect an optical signal response.

Still further in accordance with the first embodiment, wherein the cannula body has a depression radially recessing therefrom, the depression having a radial depth at least equal or greater than a diameter of at least one of the plurality of optical fibers, the fiber ends of the optical fibers being in the depression.

Still further in accordance with the first embodiment, the cannula body includes: an inner cannula having an inner sample receiving window and having an inner diameter; and an outer cannula having an outer sample receiving window and having an outer diameter, the outer diameter being larger than the inner diameter of the inner cannula, the outer cannula being adapted for receiving the inner cannula along a longitudinal axis thereof in such a way that the inner cannula is rotatable relative to the outer cannula at least about the longitudinal axis; wherein the biopsy device is an open configuration when the inner sample receiving window and the outer sample receiving window are aligned with one another and the biopsy is a closed configuration when the inner sample receiving window and the outer sample receiving window are not aligned with one another.

Still further in accordance with the first embodiment, the exterior portion of the cannula body on which is mounted the plurality of optical fibers is an exterior portion of the outer cannula, and wherein the plurality of optical fibers have a biocompatible protective disposed thereon.

In accordance with a second embodiment, there is provided a spectroscopic biopsy system for providing guidance to a mechanical biopsy procedure, the system comprising: the biopsy device in accordance with the first embodiment; at least one light generator optically coupled to at least one of the plurality of optical fibers for providing the optical signal thereto; at least one light detector optically coupled to at least one of the plurality of optical fibers for detecting the optical signal response and for generating optical signal response data associated to the optical signal response; a computing device operatively connected to at least one light detector, adapted for receiving the optical signal response data from at least one light detector and adapted for determining an optical property of the surrounding tissue based on the optical signal response data; and an output device operatively connected to the computing device for displaying the determined optical property which is to be used in the guidance of the mechanical biopsy procedure.

Further in accordance with the second embodiment, at least one light generator comprises a broadband light generator for illuminating the surrounding tissue with the optical signal comprising broadband light and wherein at least one light detector is a spectrometer for detecting the optical signal response, the determined optical property being indicative of diffuse reflectance occurring in the surrounding tissue.

Still further in accordance with the second embodiment, the computing device is further adapted to determine if the surrounding tissue comprises hemoglobin when the diffuse reflectance of the optical signal response has at least one of an increased absorption and an hemoglobin spectral signature.

Still further in accordance with the second embodiment, at least one light generator comprises a fluorescence excitation generator for illuminating the surrounding tissue with the optical signal comprising fluorescence excitation light and wherein at least one light detector is a spectrometer for detecting the optical signal response, the determined optical property being indicative of fluorescence occurring in the surrounding tissue.

Still further in accordance with the second embodiment, a fluorescence excitation filter is optically coupled to the at least one detector for filtering the fluorescence excitation wavelength.

Still further in accordance with the second embodiment, the computing device is further adapted to determine if the surrounding tissue comprises optical markers when the fluorescence of the optical signal response has at least one of an increased intensity in the emission spectrum of the optical markers or a fluorescence signature.

Still further in accordance with the second embodiment, the at least one light generator comprises a near infrared light generator for illuminating the surrounding tissue with the optical signal comprising near infrared light and wherein the at least one light detector is a spectrometer, the optical property being of Raman scattering occurring in the surrounding tissue.

Still further in accordance with the second embodiment, a near infrared light filter is optically coupled to the at least one detector for filtering the near infrared light from the optical signal response.

Still further in accordance with the second embodiment, the optical coupling between at least one light generator and the at least one of a plurality of optical fiber includes a first optical multiplexer and at least one optical switch controllable to prevent transmission of the light along a corresponding one of the at least one of the plurality of optical fibers; wherein the optical coupling between at least one light detector and the at least one of the plurality of optical fibers includes a second multiplexer and the at least one optical switch controllable to prevent transmission of the light along a corresponding one of at least one of the plurality of optical fibers; wherein the computing device is adapted to provide the optical signal along only one of the plurality of optical fibers using the at least one optical switch, the computing device being further adapted to detect one of a plurality of partial optical signal responses from each one of the at least one of the plurality of optical fibers using the at least one optical switch, the computing device being further adapted to determine the optical property based on the plurality of partial optical signal responses.

Still further in accordance with the second embodiment, said detection of the plurality of partial optical signal responses for each one of the at least one of the plurality of optical fiber is performed sequentially in time, the computing device being further adapted to construct a tomogram representative of the optical property distribution in the surrounding tissue based on said pluralities of partial optical signal responses.

In accordance with a third embodiment, there is provided a method for obtaining a tomogram of a tissue volume using a biopsy device provided in the tissue volume, the method further comprising the steps of: illuminating the tissue volume with an optical signal using at least one of a plurality of optical fibers being provided on an exterior portion of the biopsy device; detecting a plurality of partial optical signal responses associated with the other ones of the plurality of optical fibers; associating each one of the plurality of partial optical signal responses to a radial portion of the tissue volume surrounding the probing region of the cannula body; repeating said illuminating, said detecting and said associating for each one of the plurality of optical fibers and obtaining a radial distribution of partial optical responses; and processing the tomogram of the tissue volume based on said radial distribution of partial optical responses and on calibration data.

Further in accordance with the third embodiment, updating the processed tomogram such that a new tomogram is processed when the biopsy device moves in the tissue volume; and outputting a three dimensional tomogram based on the at least one previously processed tomogram obtained.

Still further in accordance with the third embodiment, the optical signal comprises broadband light signal, the plurality of partial optical signals being indicative of reflectance in the tissue volume, the tomogram being indicative of the presence of hemoglobin in the tissue volume.

Still further in accordance with the third embodiment, the optical signal comprises a fluorescence excitation signal, the plurality of partial optical signals being indicative of fluorescence, the tomogram being indicative of the presence of optical markers in the tissue volume.

Many further features and combinations thereof concerning the present improvements will appear to those skilled in the art following a reading of the instant disclosure.

### BRIEF DESCRIPTION OF THE DRAWINGS

In the figures,

FIG. 1 is a schematic side view of a biopsy device in accordance with a first embodiment of the present disclosure, with a distribution of optical fibers on a portion of the outer cannula;

FIG. 2 is a sectional view of the biopsy device of FIG. 1;

FIG. 3A is a schematic longitudinal section of the biopsy device of FIG. 1, in a closed configuration;

FIG. 3B is a schematic longitudinal section of the biopsy device of FIG. 1, in an open configuration;

FIG. 3C is a schematic longitudinal section of the biopsy device of FIG. 1, in a suction mode;

FIG. 4 is a perspective view of a biopsy device in accordance with a second embodiment of the present disclosure, with a distribution of optical fibers on the outer cannula;

FIG. 5 is a schematic longitudinal section of the biopsy device of FIG. 4;

FIG. 6 is a sectional view of the biopsy device of FIG. 4;

FIG. 7 is a table showing dimensional values that may be used for the biopsy device of FIG. 4;

FIG. 8 is a perspective view of a biopsy device in accordance with a third embodiment of the present disclosure, with optical fibers within an inner cavity;

FIG. 9 is a schematic longitudinal section of the biopsy device of FIG. 8;

FIG. 10 is an exterior view of the biopsy device of FIG. 8;

FIG. 11 is a perspective view of a biopsy device in accordance with a fourth embodiment of the present disclosure, with a motorized mirror;

FIG. 12 is a schematic longitudinal section of the biopsy device of FIG. 11;

FIG. 13 is a schematic view of a first example of a spectroscopy system in accordance with the present disclosure;

FIG. 14 is a schematic view of a second example of a spectroscopy system in accordance with the present disclosure;

FIG. 15 is a schematic cross sectional view of the biopsy device of FIG. 4 showing the number of each of the detection fibers;

FIG. 16 is a flow chart of an example of a reconstruction algorithm;

FIG. 17A is a graph of the normalized intensity versus the number of each of the optical fibers on the outer cannula of the biopsy device of FIG. 4, with data obtained when the biopsy device is in an homogeneous medium (homogeneous sinogram) and in an heterogeneous medium (heterogeneous sinogram);

FIG. 17B is a graph of the difference between the homogeneous data and the heterogeneous data of FIG. 17A versus the number of each of the optical fibers on the outer cannula of the biopsy device of FIG. 4;

FIG. 18A shows an example of a reconstructed absorption graph; and

FIG. 18B shows an example of a tomogram graph obtained using a reconstruction algorithm.

### DESCRIPTION OF THE PREFERRED EMBODIMENTS

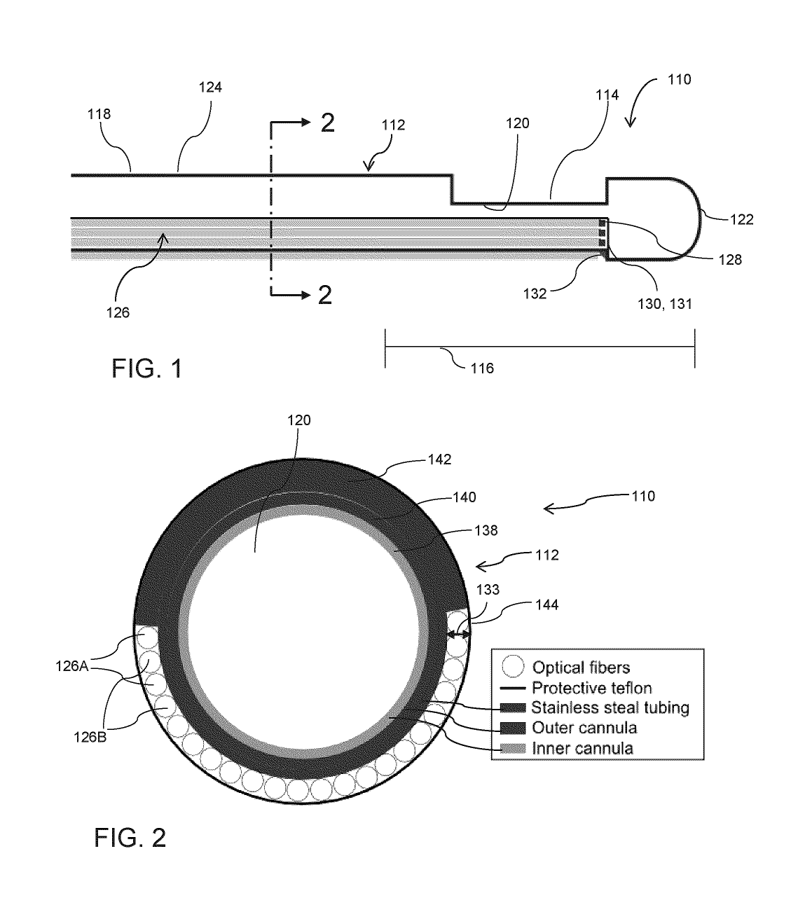

Referring to the drawings and more particularly to FIGS. 1 to 3C, a biopsy device in accordance with a first embodiment is generally shown at 110. The biopsy device 110 is an integrated optical biopsy needle that may provide information to reduce the risk of misdiagnoses and hemorrhages. The devices of the present disclosure may be used to detect and quantify tumor-specific endogenous or exogenous optical biomarkers (fluorescence and/or reflectance and/or inelastic scattering—i.e. Raman spectroscopy) locally or in a tomographic manner using an optical image reconstruction algorithm. A possible optical marker that may be used with the devices of the present disclosure is the fluorescent molecule protoporphyrin IX (PpIX), which is known to accumulate in the brain as well as other tumor types following administration of the endogenous prodrug 5-aminolevulenic acid (ALA). Concurrent with this, a structural imaging technique may be used to detect anatomical landmarks that can be used to further improve the accuracy and safety of the procedure by, e.g., sensing and localizing blood vessels and thereby prevent rupture and avoid complications related to hemorrhages.

Referring to FIG. 1, a first embodiment of the biopsy device 110 comprises a cannula body 112 with a sample receiving window 114 defined in a probing region 116. At least some of the edges defining the sample receiving window 114 may be cutting edges, such that manipulations of the device 110 may allow samples to be cut with the cutting edges. The cannula body 112 has an inner cavity 120 accessible by the sample receiving window 114 for receiving a sample of tissue, for instance. The probing region 116 of the cannula body 112 is at the distal end of a tubular body 118 and extends axially along a longitudinal axis thereof. In the embodiment shown in FIG. 1, the sample receiving window 114 is disposed on a lateral side of the tubular body, and within the probing region 116. Although the probing region 116 is shown to extend to a tip portion 122 of the tubular body 118, it can also extend to a position spaced from the tip portion 122.

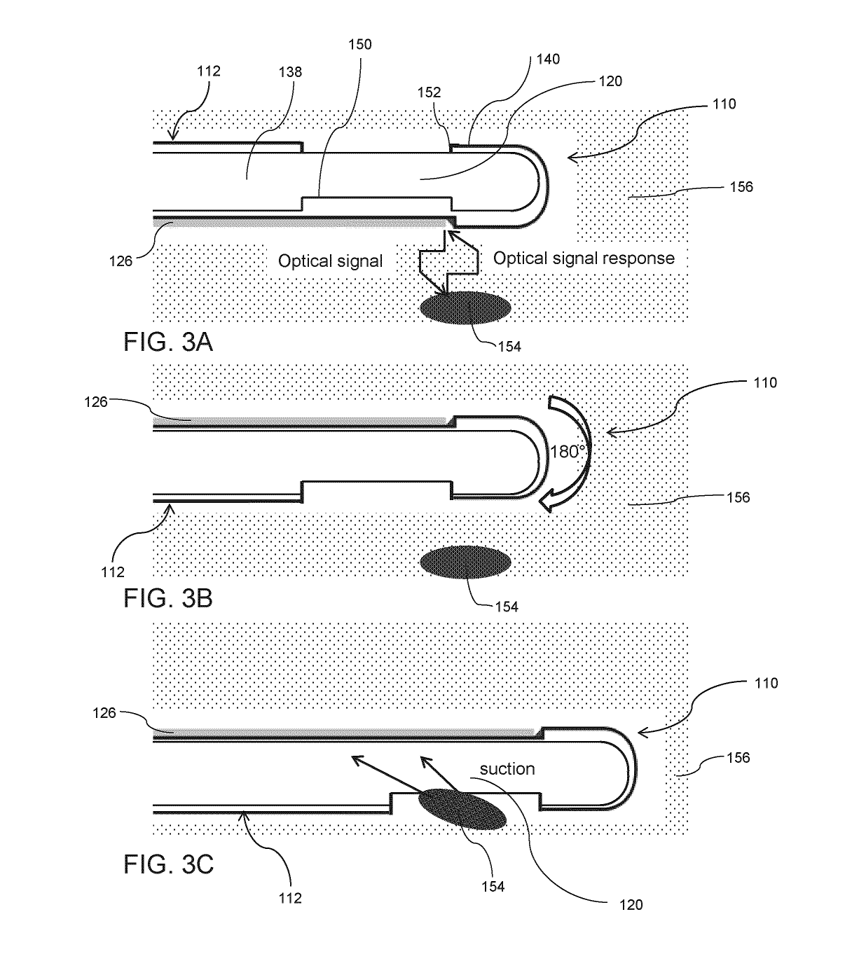

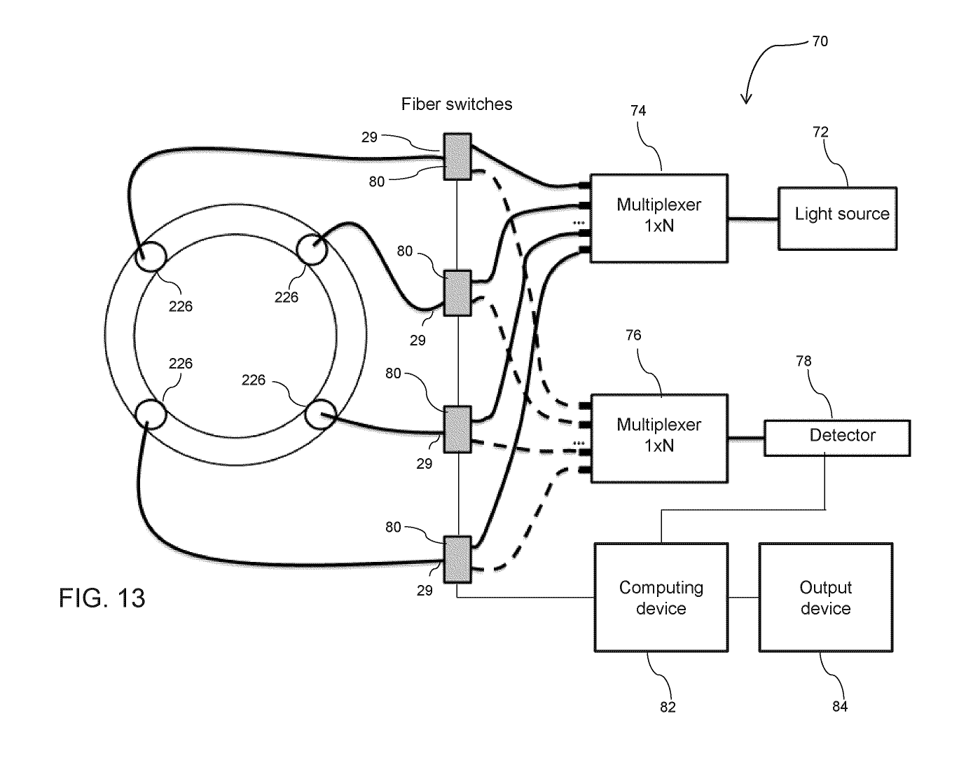

The first embodiment of device 110 has optical fibers 126 mounted along an exterior portion 124 of the tubular body 118. In this example, the optical fibers extend longitudinally along a circumferential part of the tubular body 118, opposite to the circumferential part having the sample receiving window 114. Each of the optical fibers has a fiber end 128 which extends in the probing region 116 and is axially aligned with the sample receiving window 114 and another fiber end (shown at 29 in FIG. 13) connectable to a light generator or a light detector. The fiber ends 128 are circumferentially (and/or radially) spaced from one another so that they may be evenly distributed along their circumference part of the exterior portion 124 of the cannula body 112, at a single axial position. In this first embodiment, the exterior portion 124 is optionally of lesser diameter than the tip portion 122 in the cannula body 112 so as to conceal the optical fibers 126 thereon, within the diameter of the tip portion 122. The exterior portion 124 has a depression 131 forming a shoulder 130 with the tip portion 122 of the cannula body 112, the depression 131 having a radial depth 133 (shown at FIG. 3) at least equal or greater than a diameter of the optical fiber. The biopsy device 110 may be easily assembled if the different parts are chosen and created adequately. It is contemplated that the use of the term “circumferentially” is not to limit the cannula body solely to a circular shape. First, a custom outer cannula 140 may be manufactured to provide the embedded exterior portion 124 with a larger tip than standard outer cannulas have.

Each of the fiber ends 128 is optically coupled with a redirecting surface 132, adapted to redirect light from the optical fibers 126 outwardly from the cannula body 118 either for illuminating a surrounding tissue with an optical signal or for receiving an optical signal response from the surrounding tissue. The redirecting surface 132 is aligned on an angled axis forming a non-zero angle with the longitudinal axis of the cannula body 118. In this first embodiment, the redirecting surface 132 is provided in the form of a reflective surface deposited on a face of a prism which is mounted on the cannula body 118. In other embodiments, the redirecting surface 132 can be either a reflective surface or a refractive surface. For instance, a reflective surface can be deposited on a cleaved face of each one of the optical fibers 126 or it can be an annular redirecting surface provided in the form of a polished cylindrical tube for providing a redirecting surface 132 to each one of the optical fibers 126. The redirecting surface 132 can alternatively be a refractive surface obtained with a refractive prism or by polishing a fiber end 128 which may be cleaved at a 45° angle or any suitable angle, for instance. The optical fibers 126 include illumination fibers 126A and detection fibers 126B. The illumination fibers 126A are connectable to a light generator for receiving an optical signal therefrom and for illuminating the surrounding tissue while the detection fibers 126B are connectable to a light detector for detecting an optical signal response caused by the propagation of the optical signal in the surrounding tissue. In some embodiments, the illumination fibers 126A can be used as detection fibers 126B and vice versa while in others, for instance shown at FIG. 15, illumination fibers 126A are to be used solely for illumination and detection fibers 126B are to be used solely for detection. As will be described below, combinations of the light generator and of the light detector can provide reflectance measurements, and/or fluorescence measurements and/or inelastic scattering measurements, i.e. Raman scattering, in order to determine an optical property of the interrogated tissue.

Referring to FIG. 2 which shows a cross sectional view of the biopsy device 110 of FIG. 1, the cannula body 112 comprises an inner cannula 138 receiving in an outer cannula 140. The cannula body 112 has a support tubing 142 which receives the embedded exterior portion 124. The cannula body 112 has a biocompatible protective layer 144 applied around the support tubing 142 and the optical fibers 126 for protecting the biopsy device 110 when inserted in a sample of biological tissue, for instance. In this embodiment, there are twenty-two (22) optical fibers 126, but more or less optical fibers can be provided, depending of the application.

The design of the biopsy device 110 may be suited for producing images of the tissue surrounding the device 110 in order to detect the tumor core, and the blood vessels nearby. Out of all the fibers 126, some may be used for diffuse optical tomography (DOT), while others may be used for fluorescence or Raman spectroscopy. In the embodiment of the device 110 with twenty-two (22) optical fibers 126, twenty (20) may be used for DOT and fluorescence, while the other two (2) may be used for Raman spectroscopy, these kinds of fibers 126 being used for instance in alternance. The fibers 126 diffuse light out of the biopsy device 110, but also capture light used for the detection of the tumor core and/or surrounding blood vessels. According to an embodiment, light is diffused by one of the fibers 126, and captured by all other fibers operating in the same imaging mode. The process may be repeated with all other fibers 126 to produce a complete reconstruction of the surroundings of the outer cannula of the biopsy device 110.

As seen throughout FIGS. 3A-3C, the inner cannula 138 has an inner sample receiving window 150 and the outer cannula 140 has an outer sample receiving window 152. In FIG. 3A, the biopsy device 110 is a closed configuration since the inner sample receiving window 150 and the outer sample receiving window 152 are not aligned. For instance, the operator of the biopsy device, e.g., the surgeon, may maintain the biopsy device 110 in the closed configuration while using optical fibers 126 to determine the optical property of a sample 154 of surrounding tissue 156. If the determined optical property of the sample 154 is indicative of a tumor, for instance, the operator may rotate the outer cannula 138 so that the biopsy device 110 reaches an open configuration, i.e. when the inner sample receiving window 150 and the outer sample receiving window 152 are aligned with one another as shown in FIG. 3B. While in the open configuration, the biopsy device 110 may be manipulated to remove the sample 154 of the tumor in the surrounding tissue 156 by suctioning it into the inner cavity 120 as shown in FIG. 3C, or by using cutting edges delimiting the window to remove the sample.

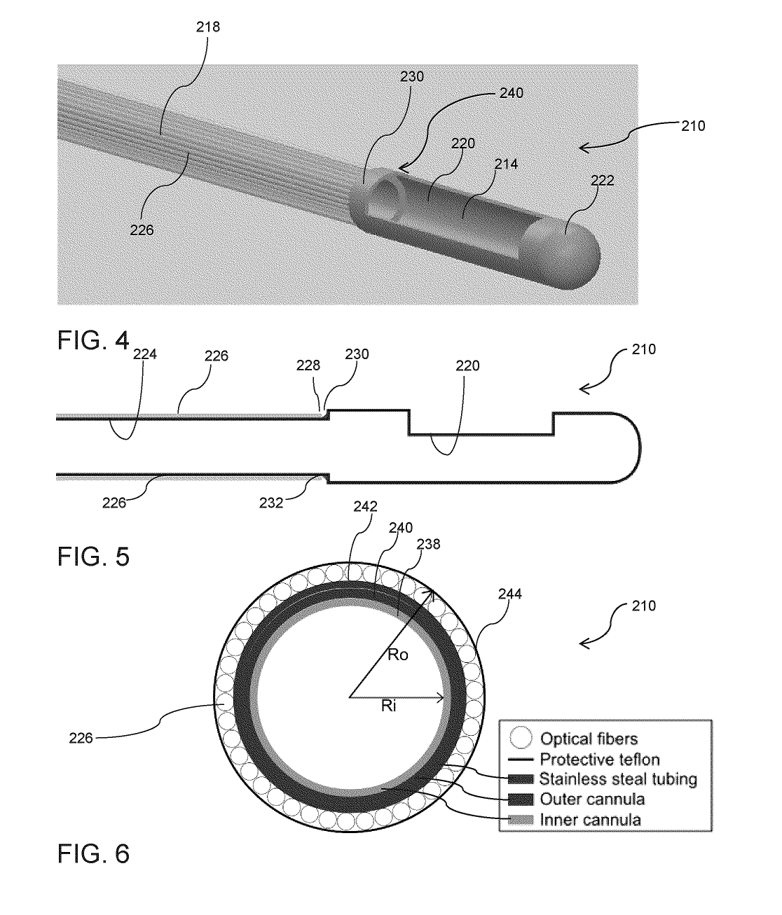

FIGS. 4-6 show a second embodiment of the device 210 which comprises an outer cannula 240 with an enlarged tip 222, with window 214. At least some of the edges defining the window 214 may be cutting edges, such that manipulations of the device 210 may allow samples to be cut with the cutting edges. The enlarged tip 222 of the outer cannula 240 is at the distal end of a tubular body 218. The enlarged tip 222 and the tubular body 218 concurrently define an inner cavity 220 of the device 210, with access for samples via the window 214.

The tip 222 is said to be enlarged, as its outer diameter is slightly greater than that of the tubular body 218, thereby defining a shoulder 230. The device 210 therefore includes numerous optical fibers 226 evenly distributed around the tubular body 218 and ending in close proximity to the shoulder 230 of the tip 222 of the biopsy device 210. In an embodiment, sixty-four (64) fibers 226 are provided, and may be protected by a biocompatible jacket 244 (FIG. 6) that can be sterilized. More or fewer of these fibers 15 may be provided on the device 110. The optical fibers 15 can be seamlessly connected to optical hardware (through, for example, SMA connectors) allowing different types of signal detection to be achieved. As shown in FIG. 5, at the distal end of the fibers 226, near the shoulder 230, a small mirror 232 may be machined (e.g., at 45 degrees relative to a longitudinal axis of the fibers 226) in order to guide the light perpendicularly to the device 210 (FIG. 5). The small mirror 232 is one of numerous configurations to guide light, reference being made to device 110 for others.

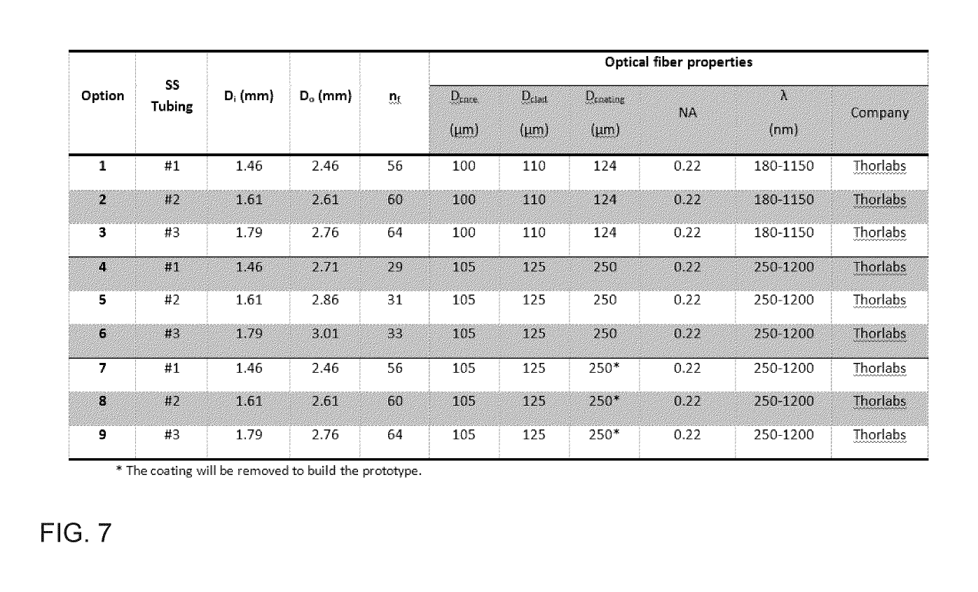

The biopsy device 210 may be easily assembled if the different parts are chosen and created adequately. First, a custom outer cannula 240 (e.g., diameter ˜1.9 mm) may be manufactured to provide the enlarged tip 222 than standard outer cannulas do not have. The mirror 232 can be made by polishing a cylindrical aluminium tube and then inserted outside the outer cannula (it can rest against the shoulder 230). In order to have a tight assembly of the fibers 226 around the tubular body 218, they may be glued beforehand on a support tubing 242 (FIG. 6) that can fit perfectly around the tubular body 218 of the device 210. To do so, it is considered to create a vertical setup that can hold the tubular body 218 and the fibers 226. Once everything holds vertically (e.g., with the help of various weights), the fibers 226 (22 fibers, 25 μm core diameter, 245 μm coating diameter, NA=0.1, Thorlabs) may be glued to the cylinder in a circumferential, equidistant and evenly distributed manner using an optical adhesive. The same adhesive may be used to hold the support tubing 242, the redirecting surface 232 (e.g. a 90° mirror or prism, ensuring illumination normal to the biopsy device) and the outer cannula 240 together. Once these three components are fixed, a protective layer 244 may be added. In an embodiment, the protective layer 244 may consist of a heat-shrink Teflon tube, which can be biocompatible and sterilizable. Referring to FIG. 6, an inner cannula 238 (e.g., diameter ˜0.8 mm) is shown within the inner cavity of the outer cannula 240 and movable with respect to the outer cannula 240. Although the above mentioned manufacturing technique is directed to the biopsy device 210, it may also be used for the biopsy device 110 or any suitable embodiment of the biopsy device in accordance with the present disclosure. In FIG. 7, a table showing dimensions for the biopsy device 210 are provided. The dimensions are provided by way of example, and are not to be interpreted as being !imitative. The dimensions are for an approximate thickness of the inner and outer cannula 238, 240 of 250 microns, and three different diameters for the support tubing 242.

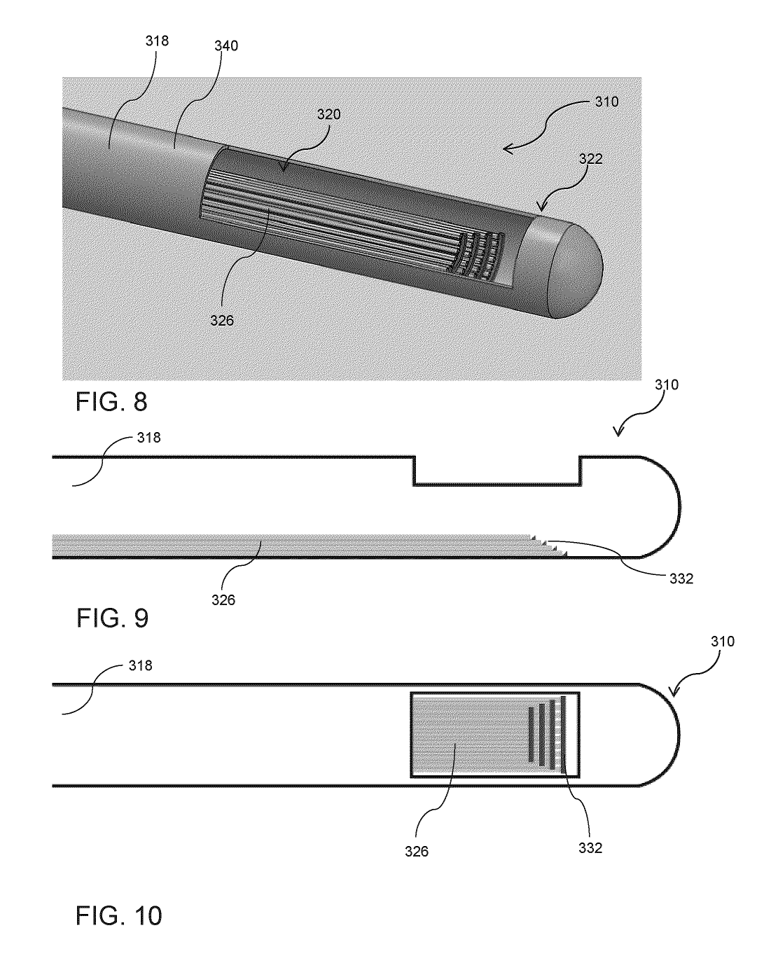

Referring to FIGS. 8 to 10, a biopsy device in accordance with a third embodiment is shown at 310. The device 310 comprises an outer cannula 340 with a tip 322, with window 314. The tip 322 of the outer cannula 340 is at the distal end of a tubular body 318, and integral therewith in providing a continuous surface of uniform diameter, until the very end is reached. The tubular body 318 has an inner cavity 320, with access for samples via the window 314.

Optical fibers 326 are positioned within the inner cavity 320 of the tubular body 318, and are arranged in rows forming steps. In the illustrated embodiment, the fibers 326 are at the bottom of the inner cavity 320 and may be protected by an optically clear material. Here, all fibers 326 could do DOT and fluorescence. This biopsy device 310 also uses machined mirrors 332 (e.g., machined at 45°) to image perpendicularly to a longitudinal axis of the device 310.

To achieve a structure of the device 310 as shown in FIGS. 8-10, a mold may be created that has the shape of the bottom row. By doing so, a support would be in place for the fibers 326 and mirror 332 so that they may be glued together. Afterwards, the support could be glued to a flexible sheet that would be used to insert the assembly of fibers 326 inside the tubular body 318. The second, third and fourth row (etc.) would all be assembled the same way. The fibers 326 and mirrors 332 would be placed using a mold that would set their places in relation to the previous row. Once the assembly done, the sheet can be inserted and glued inside the inner cavity 320 of the tubular body 318. To make sure the fibers 326 are protected, Teflon tubing protective layer may be placed above them, such as for the device 110 of FIGS. 1-3 and for the device 210 of FIGS. 4-6.

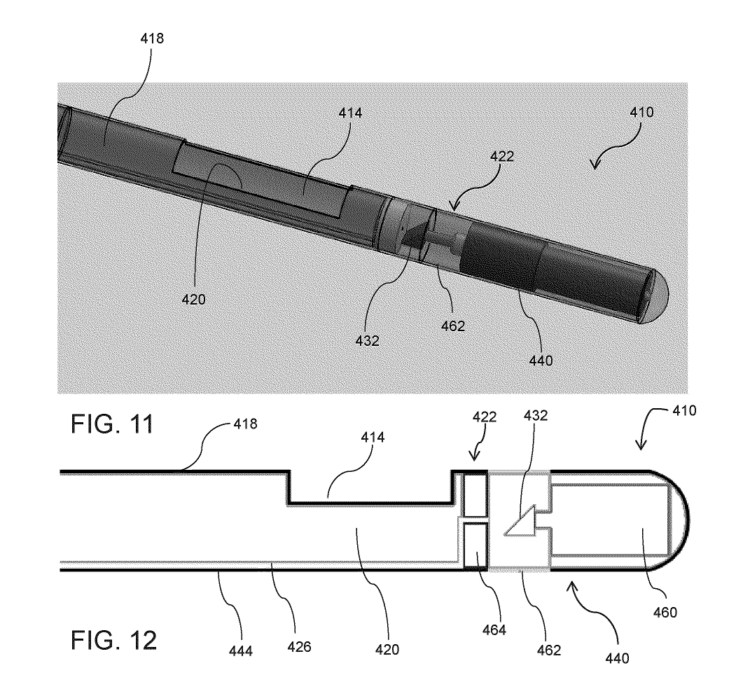

Referring to FIGS. 11-12, a biopsy device in accordance with a third embodiment is shown at 410. The device 410 comprises an outer cannula 440 with a tip 422, with window 414. The tip 422 of the outer cannula 440 is at the distal end of a tubular body 418, and integral therewith in providing a continuous surface of uniform diameter, until the very end is reached. The tubular body 418 has an inner cavity 420, with access for samples via the window 414.

The device 410 may use only one optical fiber 426 and mirror 432, as well as one micromotor 460, with the center of the mirror 432 glued to the motor shaft. The optical fiber 426 carries the signal up to the mirror 432 which sends it perpendicularly to the device 410. A Plexiglas window 462 is added to the tip 422 of the outer cannula 440 so the signal may go through it. The rotation of the micromotor 460 allow a 360-degree rotation of the optical signal, thus providing a complete image of the surrounding tissue. With this design of the device 410, it is possible to integrate various imaging modalities (DOT, optical coherence tomography (OCT), Raman spectroscopy and fluorescence). The fiber 426 can be at the center of a circular support 464 that can ensure its alignment with the mirror 432. All these components can be glued manually inside the inner cavity 420 and can then be protected by a protective Teflon jacket 444.

Referring to FIG. 13, there is illustrated a first example of a spectroscopic biopsy system 70 for providing guidance to a mechanical biopsy procedure for use with anyone of the biopsy device 110, 210, 310, and 410, although it is shown with the biopsy device 210 having sixty-four (64) optical fibers 226 as an example. The light generator is shown at 72, and produces an optical signal directed to first optical multiplexer 74, that can direct light to the illumination fibers 226A for illuminating the surrounding tissue. Upon illumination by the optical signal, an optical signal response is measured with the detection fibers 226B. The detection fibers 226B direct the optical signal response to second optical multiplexer 76, that can direct the optical signal response to a light detector shown at 78. The light detector is a spectrometer that analyzes the spectrum of the optical signal response. For instance, the spectrometer generally provides data indicative of intensity counts versus wavelengths. The first and second optical multiplexers 74, 76 are respectively characterized as 1×N, where N refers to the number of illumination or detection optical fibers connected thereto. The optical fibers 226 are controllable by optical switches 80 which can couple the multiplexers 74, 76 to the optical fibers 226, to send the light from the light generator 72, and collect the light to be sent to the light detector 78. The light detector 78 is generally adapted to generate an optical signal response data associated to the optical signal response. The optical signal response data is to be provided to a computing device, shown at 82, which can be used to control the optical switches 80, the light generator 72, the light detector 78 and the multiplexers 74, 76, for instance. The computing device 82 is adapted to determine an optical property based on the optical signal response data received from the light detector 78. Once the optical property is determined, it can be displayed to a surgeon, for instance, on an output device 84 operatively connected to the computing device 82. The spectroscopic biopsy system 70 is one of numerous possible options which can be suited for the detection of a particular analyte or a combination thereof. Indeed, the light generator 72 can include a combination of multiple types of light generators which are each suited for a particular use and the light detector 78 can include a combination of filters associated to multiple types of light detectors, e.g. one high-resolution spectrometer (e.g., for Raman spectroscopy) and one lower-resolution spectrometer (e.g. for reflectance and fluorescence).

Using the system 70, tomographic data can be acquired by performing the exemplary steps of: providing the biopsy device in the tissue volume; illuminating the tissue volume with the optical signal by providing the optical signal to one of the plurality of illumination fibers; detecting a plurality of partial optical signal responses associated with corresponding ones of the plurality of detection fibers; associating each one of the plurality of partial optical signal responses to a radial portion of the tissue volume surrounding the probing region of the cannula body; repeating said illuminating, said detecting and said associating for each one of the plurality of illumination fibers and obtaining a radial distribution of partial optical responses; and processing a tomogram of the tissue volume based on said radial distribution of partial optical responses and on calibration data. It can also include steps of providing the biopsy device at least at another depth of the tissue volume; and obtaining a tomogram for each one of the at least another depth at which the biopsy device is provided; and determining a three dimensional tomogram based on the at least two tomograms obtained. It is contemplated that the processed tomogram can be updated so that once the three-dimensional tomogram is obtained, it can be displayed to the surgeon, thus enabling him/her to make a decision on whether the surrounding tissue comprises blood vessels or cancer cells, for instance, and further decide to remove (or not) the sample of tissue. For instance, if the biopsy device 110 comprises 22 optical fibers 126 evenly distributed on half the circumference of the outer cannula 140 and numbered from 1^{st} fiber to 22^{nd} fiber, the optical signal is propagated into the first fiber and partial optical signal responses are detected for the 2^{nd} to the 22^{nd} fiber. Then, the optical signal is propagated into the 2^{nd} fiber and partial optical signal responses are detected for the 3^{rd} to the 22^{nd} and for the 1^{st} fiber. Repeatedly performed, this detection procedure can provide 22 sets of 21 optical signal responses that can be used to produce a tomogram using a reconstruction algorithm processed by the computing device 82. In other embodiments, there may be optical fibers 126 solely used as illumination fibers and other optical fibers 126 used solely as detection fibers.

The various components used in the system 70 are dependent on the contemplated use. For instance, the lighting system 70 may produce spectroscopic diffuse reflectance signals to detect hemoglobin (localized in blood vessels) based on increased tissue absorption and/or the spectral signature and concentration of oxygenated and deoxygenated hemoglobin. In such a case, the light generator 72 may be a white-light lamp and the light detector 78 may be a portable spectrometer with detection sensitivity in the visible and NIR parts of the electromagnetic spectrum.

For tissue characterization, the lighting system 70 may perform the spectroscopic fluorescence detection of an exogenous marker (e.g., a fluorophore). In such cases, the light generator 72 may be single wavelength or tunable laser or light-emitting diode (LED), and the light detector 78 may be a portable spectrometer with detection sensitivity in the visible and NIR parts of the electromagnetic spectrum. The detection may include suitable filters in order to detect the fluorescence emission while filtering the fluorescence excitation signal. In the case of spectroscopic fluorescence detection of tissue auto-fluorescence, the requirement for tunability at excitation might render a signal much more specific and as such might be an absolute requirement. Spectroscopic detection of Raman spectra (inelastic scattering) is also considered, using a wavelength-stabilized NIR laser and a high-sensitivity spectrometric detector.

The system 70 can be used to acquire hyperspectral tomographic data using the biopsy device 210, for instance, as it could be also either one of the biopsy device 110, 310 and 410. The tomographic data are computed from the reflectance spectra from the light generator 72 being provided as a white-light source for generating broadband light into the illumination optical fibers 226. Although a design based on 22 fibers is presented here, detailed simulation studies may be conducted to determine the optimal number and configuration of source and detector fibers, so it can be more than 22 fibers or less than 22 fibers. The illumination/detection and control system can have a 20 W tungsten halogen light source (e.g., Ocean Optics) connected to a software-controlled optical multiplexer (e.g., Ocean Optics).

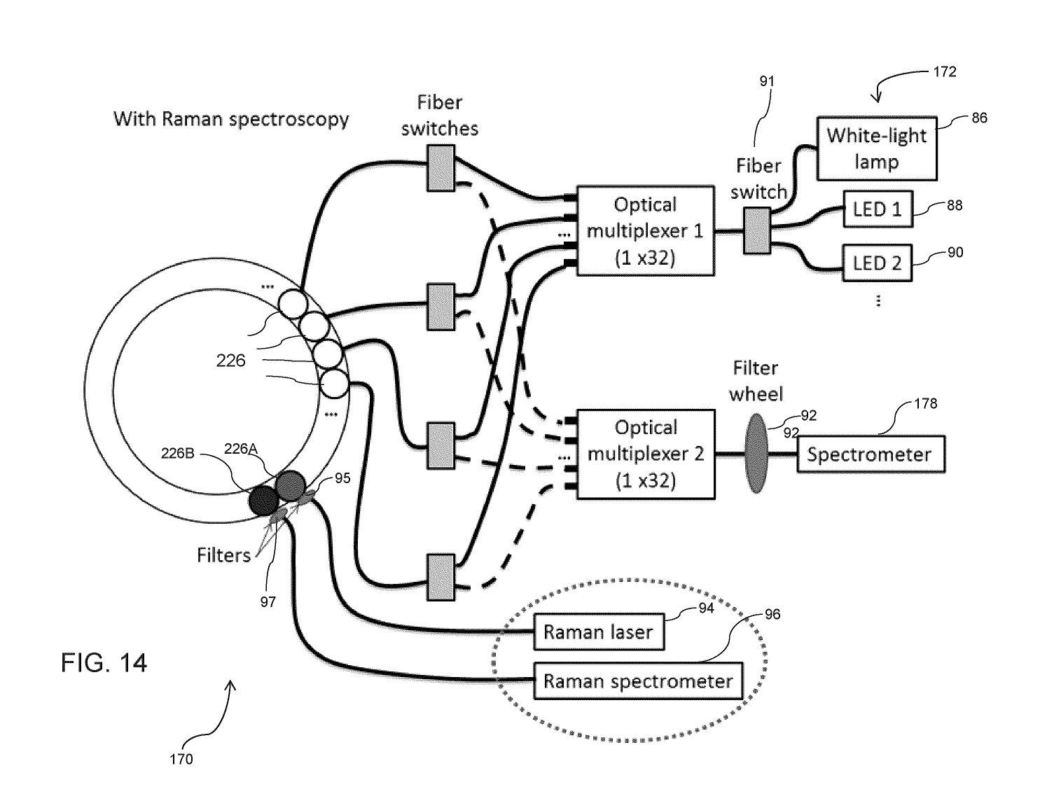

Referring to FIG. 14, there is illustrated a second example of a spectroscopic biopsy system 170 for providing guidance to a mechanical biopsy procedure for use with anyone of the biopsy device 110, 210, 310, and 410, although it is shown with the biopsy device 210 having sixty-four (64) optical fibers 226. The light generator 172 includes a white-lamp source 86 for providing broadband light, a first LED 88 for providing fluorescence excitation light comprising a first fluorescence excitation wavelength (e.g., 405 nm) and a second LED 90 for providing fluorescence excitation light comprising a second fluorescence excitation wavelength (e.g., 455 nm). The three light generators 86, 88, 90 are connected to a second fiber switch 91 for illuminating tissue surrounding the biopsy device 110 with the optical signal generated by either one of the light generators 86, 88, and 90. The light detector 178 may have a filter wheel 92 connected thereto. The filter wheel 178 comprises a plurality of filters each designed either for a corresponding one of the light generators 86, 88, 90. In this example, an illumination fiber 226A is connected to a near infrared (NIR) laser 94 (e.g., a Raman laser at 785 nm) via a first filter 95 (e.g., a narrow high-rejection, large attenuation band-pass filter) to minimize the propagation of the (non tissue-specific) inelastic signature of the fiber (between the NIR laser 94 and the first filter 95) into the detection fiber 226B which is connected to a high-resolution spectrometer 96 (e.g. a Raman spectrometer) via a second filter 97 (e.g., a high-pass filter). The first filter 97 may minimize the signal contribution associated with tissue elastic scattering (typically orders of magnitude larger than the inelastic contribution that is targeted for detection) and to further reduce the propagation of the (non tissue-specific) inelastic signature of the fibers. For instance, for fluorescence and Raman spectroscopy, the filters 92, 95 and 97 may be disposed in the light path, either at the fiber ends 128 of the fibers 126—close to the redirecting surface 132, or outside of the biopsy device, closer to the light generator and light detector. Challenges associated with Raman spectroscopy are known in the art. Indeed, it is known that an elastic (Rayleigh) scattering signal is several orders of magnitude higher than a non-elastic (Raman) signal, therefore the first and second filters 95, 97 have to be carefully chosen for suitably filter the elastic (Rayleigh) scattering signal. Examples of methods for obtaining such filters may be described in US Patent Application Publication Serial No. US 2012/0236303 A1 to Marple et al. and in U.S. Pat. No. 8,175,423 to Marple. In another embodiment, the Raman laser and the Raman spectrometer are provided respectively with the light generator 172 and with the light detector 178. The computing device and the output device are not illustrated in FIG. 14 for clarity purposes.

The biopsy devices 10, 210, 310 and 410 may reduce the risks associated with stereotactic brain needle biopsies (BNB) by combining fluorescence and/or Raman spectroscopy with a novel tomographic approach for imaging with diffuse light. On one hand, the tomographic imaging allows a 360-degree reconstruction of the absorption coefficient near the device, thus indicating the presence of blood vessels. On the other hand, fluorescence spectroscopy using, for instance, protoporphyrin IX and/or Raman spectroscopy can help identify cancer tissue. Both these techniques may effectively reduce the number of biopsies required as well as the risks of BNB-related hemorrhages.

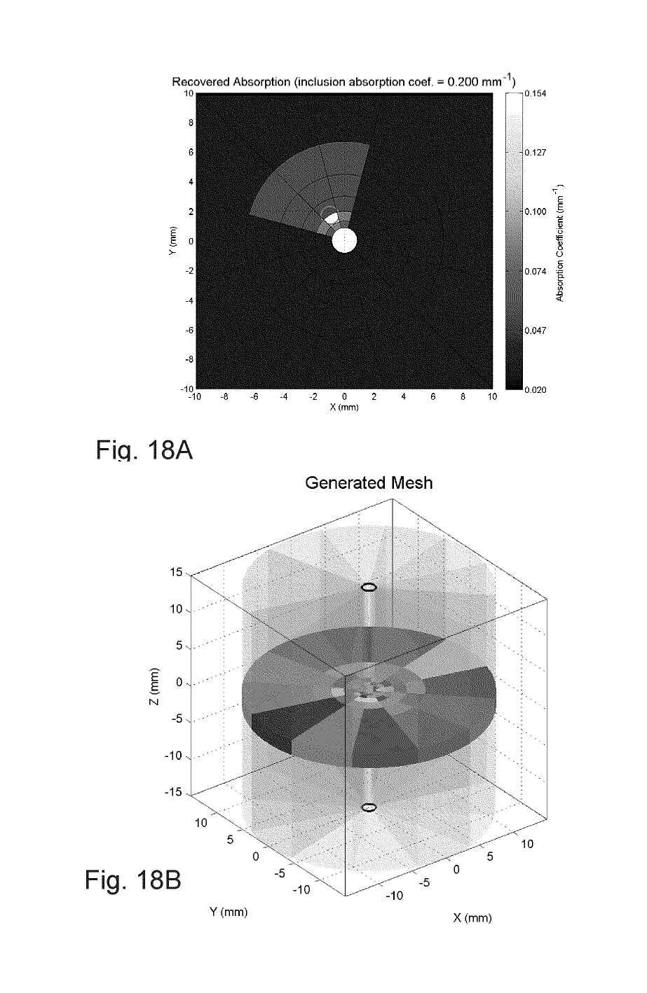

The reconstruction algorithm (as presented below) is performed at a single wavelength (and so FIGS. 17A to 18B show single-wavelength reconstructions), although it is contemplated that it may be performed all wavelengths acquired with the light detectors. Indeed, multiple wavelengths can be used for further improving reconstruction accuracy. Instead of reconstructing the absorption coefficient, μₐ, at one wavelength, the known absorption spectra of oxy-hemoglobin and deoxy-hemoglobin (the dominating absorbers in brain) can be used to reconstruct their volumetric concentrations using the formula:

μₐ(λ)=c_{Hb}[SₜO₂μₐ(λ)^{oxyHb}+(1−SₜO₂)μₐ(λ)^{deoxyHb}].

This may improve the specificity (to haemoglobin: a surrogate for blood vessels) of the reconstructions.

The biopsy device of the present disclosure allows identification (and/or classification) and localization of blood vessels, based on the distinctive optical absorption signature of hemoglobin in the local diffuse reflectance spectrum.

The core PpIX quantification technique is already established. Hence, a remaining challenge is blood vessel detection/localization. The present disclosure involves algorithms to estimate accurately the blood vessel distance from the needle.

According to the present disclosure, the reflectance spectra at multiple points around the circumference of the biopsy device is used as input to a full short-range, multi-spectral optical tomography reconstruction algorithm, and preliminary results support the fact that blood vessels can both be detected and their location estimated. The fluorescence and reflectance measurements can be initiated 1 cm from a contrast-enhancing region observed on a pre-operative scans and the profile can then be sampled frequently (every ˜2 mm, depending on the rate of needle advance and data processing time) through the enhancing region and 1 cm beyond, which can identify the location where a tissue specimen is best taken (based on C_{PpIX} and the intrinsic optical biomarkers), and show if significant blood vessels (>˜0.5 mm diameter) are present within (at least) 2 mm from the needle track.

In the biopsy device of the present disclosure, the small diameter of the inner cannula (e.g., <2 mm) may limit the extent of tissue that can be aspirated and cut. Thus, blood vessel detection within this distance is required to reduce the risk of hemorrhage. The optical contrast (spatial and spectral) due to blood absorption may be used to detect these vessels and determine their location and distance, as the optical contrast of blood vessels in a glioblastoma multiforme (GBM) can be as high as 10:1 at 470 nm and 30:1 at 705 nm. Spectral data can locate vessel depths, since superficial vessels are more clearly evident at 470 nm than at 705 nm due to the larger tissue sampling depth at the longer wavelength. Thus, the location of blood vessels relative to the light sources 72 and detectors 78 can cause quantifiable changes to both the magnitude and the shape of the detected diffuse reflectance spectra measured by the interstitial probe.

Two methods are considered for blood vessel detection and localization. First, a multi-spectral ‘near-field’ mesoscopic optical tomography (MOT) approach (Method 1, below) is used to reconstruct 3D HbR and HbO2 images around the biopsy device from numerous spatially and spectrally resolved measurements (e.g., 256) at each of different increment of wavelengths (e.g., 2-10 nm).

Second, a non-tomographic approach (Method 2, below) may be used where distortion in the hemoglobin spectrum caused by intervening tissue from only a few measurements can be exploited to estimate locally (rather than tomographically) the distance of vessels from the probe surface. This approach may be less sensitive to noise and error propagation relative to MOT. Both techniques can use data from the same hyperspectral detection system. The Method 2 is relatively more straightforward than the Method 1 as no tomographic reconstruction is required.

The following relates to the tomographic approach in which a tomogram is to be computed from the plurality of partial optical signal responses. It describes an example of a reconstruction algorithm which can be used concurrently with the biopsy device 110, 210, 310 or 410 in reflectance and fluorescence configuration. In optical microscopy, the detected light is minimally scattered leading to very high (μm) spatial resolution and little penetration depth (˜100-200 μm). Conversely, diffuse optical tomography (DOT) for large-volume imaging exploits scattered (diffuse) light and relies on a light-transport model to account for the statistical spread of photon paths, resulting in relatively poor resolution (typically several mm's). The present disclosure proposes a mesoscopic approach, in which the sampling volume and resolution lie between those of microscopy and DOT. Unlike the conventional DOT geometry, the imaging volume is exterior to the illumination fibers and to the detection fibers, whose fiber ends' separation is typically in the order of ˜0.5-5 mm, given the dimensions of the biopsy device of the present disclosure. For these distances, the diffusion approximation may not be appropriate; therefore, a reconstruction algorithm (involving Monte Carlo simulations) being adapted to the detection geometry of the biopsy device of the present disclosure is used (including boundary conditions), wherein the algorithm is adapted to determine an optical property of a tissue surrounding the biopsy device, in the visible range where absorption is comparable to scattering.

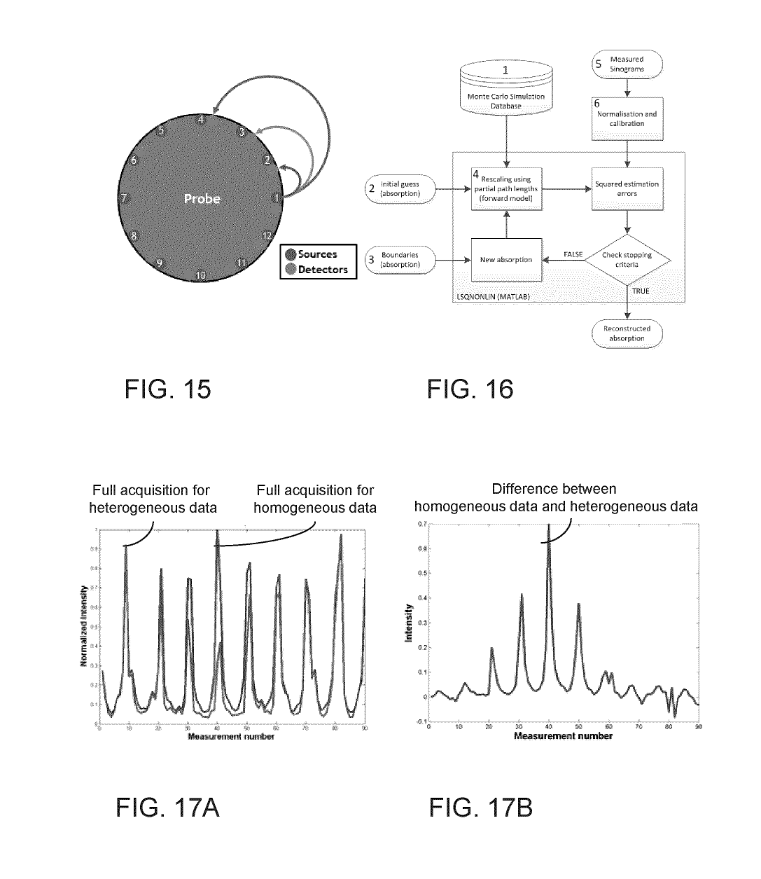

Referring to FIG. 16, there is provided an exemplary flow chart of a reconstruction algorithm for use with the biopsy device 110, 210, 310 or 410, for instance. Say that an experiment is to be performed with the biopsy device 210 shown at FIG. 15. In such circumstances, the biopsy device 210 comprises twelve (12) pairs of illumination and detection fibers 226A, 226B distributed on the circumference of the outer cannula 240 and numbered from 1^{st} detection fiber to 12^{th} detection fiber, the optical signal is propagated into a first illumination fiber (not numbered) and partial optical signal responses are detected for the 1^{st} to the 12^{th} fiber. Then, the optical signal is propagated into a 2^{nd} illumination fiber (not numbered) and partial optical signal responses are detected for 1^{st} to the 12^{nd} fiber. Repeatedly performed sequentially in time, this detection procedure can provide 12 sets of optical signal responses (or radial distribution of partial optical signal responses) that can be combined to produce a sinogram (examples of homogeneous and heterogeneous sinograms are shown at FIGS. 17A and 17B) which is to be further used to produce a tomogram using the reconstruction algorithm processed by the computing device 82. For calibrating the biopsy device in accordance with the present disclosure, a sinogram is to be acquired while the biopsy device lies in an homogeneous medium, hereinafter referred to as a ‘homogeneous sinogram’. Once the homogeneous sinogram is properly acquired, each of the illumination and detection fibers can be calibrated to have a corresponding gain or performance. This step of calibration is included in block 6 of the flow chart of FIG. 16. In using the biopsy device into an heterogeneous medium such as biological tissue, for instance, any inhomogeneities may absorb light in a manner indicative of said inhomogeneities. For example, if a blood cell distribution is located near the 12^{th} detection fiber of the biopsy device 110, the detected light at that position may be changed compared to the light that would have been detected in an homogeneous medium. Therefore, the reconstruction algorithm provided herein compares measured heterogeneous sinograms with simulated heterogeneous sinograms in order to find a simulated heterogeneous sinogram that fits with the measured heterogeneous sinogram. FIG. 18B shows a reconstruction absorption around a biopsy device 110 based on a measured sinogram. The larger the distance between tips of the illumination and detection fibers, the deeper the detected light penetrates into the tissue volume, as shown in FIG. 15, which should allow information to be recovered up to 2 mm or more from the biopsy device 110, for instance.

More specifically, the algorithm includes: obtaining measured sinograms shown at block 5; calibrating the measured sinograms using a calibration homogeneous sinogram shown at block 6; simulating homogeneous and heterogeneous sinograms such as shown concurrently at blocks 1, 2, 3, 4; comparing the simulated sinograms with the measured sinograms until a certain criteria is met such as shown at blocks 7 and 8; and obtaining a reconstructed sinogram when the certain criteria is met such as shown at block 9. The comparing step may include a non-linear mean square method (trust region reflective algorithm) which requires initial guesses and boundaries (lower bounds and upper bounds) for parameters used in the simulation of the simulated sinograms. MATLAB functions such as LSQNONLIN may be suitable for such a comparing step.

Simulated sinograms can be obtained from the light intensity change (perturbation) due to a small absorption perturbation, Δμₐ, at wavelength A and at location (rₙ, φₙ, zₙ) from the biopsy device (or needle) (r_{needle}, φ=0, zₜₒₚ) given by:

ΔI_{m,n}^{λ}=J_{m,n}^{λ}(rₙ−r_{needle},φₙ,zₙ−zₜₒₚ)×Δμₐ(rn−r_{needle},φₙ,zₙ−zₜₒₚ), (Equation 1)
where the Jacobian, J, is a statistical average of the photon tracks for source-detector pair, m. The index, n, scores the nodes in the finite-element mesh, and J can be computed for each source-detector pair using Monte Carlo simulations, and assuming constant optical properties in the tissue volume surrounding the biopsy device, namely the scattering coefficient, μₛ, the anisotropy factor, g, and the absorption coefficient, μₐ=εC, i.e., the product of the extinction coefficient and concentration of the two main chromophores (or exogenous markers), HbR and HbO₂:

μₐ(λ)=ε_{HbR}(λ)C_{HbR}+ε_{HbO}_{₂}(λ)C_{HbO}_{₂}. (Equation 2)

Equation 1 can be written in matrix form ΔI=J ΔC, where each line corresponds to the measurement from one source-detector pair at a given wavelength, and where each row of J is the computed photon sensitivity at one node in the finite-element mesh. That is,

$$( \begin{matrix}Δ {I}_{{λ}_{1}} \\ Δ {I}_{{λ}_{2}} \\ ⋮ \\ Δ {I}_{{λ}_{n}}\end{matrix} ) = ( \begin{matrix}{ɛ}_{{HbO}_{2}}^{{λ}_{1}} {J}_{{λ}_{1}} & {ɛ}_{HbR}^{{λ}_{1}} {J}_{{λ}_{1}} \\ {ɛ}_{{HbO}_{2}}^{{λ}_{1}} {J}_{{λ}_{1}} & {ɛ}_{HbR}^{{λ}_{1}} {J}_{{λ}_{1}} \\ ⋮ & ⋮ \\ {ɛ}_{{HbO}_{2}}^{{λ}_{1}} {J}_{{λ}_{1}} & {ɛ}_{HbR}^{{λ}_{1}} {J}_{{λ}_{1}}\end{matrix} ) \times ( \begin{matrix}Δ {C}_{{HbO}_{2}} \\ Δ {C}_{HbR}\end{matrix} ) , \qquad ( Equation 3 )$$

where ΔC_{HbR} and ΔC_{HbO2} are the perturbation in hemoglobin concentration around the baseline value. The scattering is assumed constant. The parameters of interest here are the local ΔC_{HbR} and ΔC_{HbO2} values distributed around the biopsy device of the present disclosure. Equation 3 can be solved with, e.g., a conjugate-gradients algorithm, regularized to minimize noise propagation yet minimize the objective function ∥ΔC−Δ/∥², as presented in the scientific publication referenced as “H. Dehghani, M. E. Eames, P. K. Yalavarthy, S. C. Davis, S. Srinivasan, C. M. Carpenter, B. W. Pogue, and K. D. Paulsen, “Near infrared optical tomography using NIRFAST: Algorithm for numerical model and image reconstruction,” Commun Numer Methods Eng 25, 711-732 (2008)”. Despite the fact the problem is intrinsically non-linear (J is a function of the optical properties), the Jacobian may not be updated during the reconstruction, since this would be too time consuming (using Monte Carlo) for neurosurgical workflow. Although less accurate in terms of recovered contrast, the linear approximation has been shown in DOT to provide accurate localization of optical contrast. Hence, the images can be reconstructed using data acquired from a single 2D plane, while image reconstruction can be performed in 3D.

Measurement pre-processing/calibration prior to image reconstruction may be critical, and may involve a normalization step in which each recording can be converted to a perturbation with reference to the background. A simple one-step calibration process would involve acquiring a full tomographic dataset in an homogeneous optical phantom (known optical properties). Due to the acquisition's cylindrical geometry, all projections (measurements taken at a given laser injection point) should be identical: the relative gain of each detector-source pairs can be computed. The proposed algorithm exploit normalized dataset: absolute calibration is therefore not required, although achievable.

Data calibration routines and/or the use of data types other than intensity (e.g. spectral derivatives) will also be considered to handle differences in light-tissue coupling. A potential problem is the under-determined solution, e.g., a small blood vessel close to the needle may have the same optical signals as a large vessel farther away. The use of multiple spectrally-resolved measurements may resolve such ambiguities.

The simulated sinograms may be obtained via Monte Carlo simulations (MCS). These MCS are used as a light propagation model. In this example, scattering and anisotropy coefficients may be assumed constant and the optical contrast approximated to come solely from absorption. This approximation is motivated by tissue optical properties measurements we have made in human patients during neurosurgical procedures. Tomographic data can be obtained by simulating the different laser injection points with their respective detection points, with appropriate pre-defined source and detector sizes and numerical apertures. The probe geometry is modeled by a tetrahedral mesh and simulations are performed using Mesh-based Monte Carlo (MMC) by Qianqian Fang described in scientific publications “Qianqian Fang, Mesh-based Monte Carlo method using fast ray-tracing in Plucker coordinates, Biomed. Opt. Express 1(1), 165-175 (2010)” and “Qianqian Fang and David R. Kaeli, Accelerating mesh-based Monte Carlo method on modern CPU architectures, Biomed. Opt. Express 3(12), 3223-3230 (2012)”.

With respect to the simulated sinograms, photons are allowed to propagate as a packet of equivalent weight along a particular trajectory. These are called photon packets (PP). As opposed to a single photon that would be completely absorbed on the first absorption event, the PP loses its weight gradually allowing longer paths of better statistical relevance. Each MCS (for each source-detector pair and the associated large number of injected photons) outputs the partial path lengths (PPL) of detected photon packet (PP). The PPL represents the distance travelled in each region (tissue) of the tetrahedral mesh for a given PP. The intensity of a packet can be computed via the Beer-Lambert law, which allows evaluation of the attenuation of light as it propagates into an absorbing medium. As a result, the total measured intensity at a given detector is the sum of the energy of each detected photon packet:

I=Σₙ₌₁^{N}exp(−Σₖ₌₁^{K}μₐ^{k}sₖ), (Equation 5)
where I the detected intensity at a given detection point, N the number of detected photons at this point, K the number of regions, sₖ the partial path length in region k for the photon n. By recording the PPL of each PP, tomographic measurements can be rescaled for a variety of absorption coefficients: with a single dataset from a single MCS, we can obtain the sinograms for a great range heterogeneous medium, as set out at block 1 of FIG. 16.

For each illumination fibers around the periphery of the biopsy device of the present disclosure, spectrally-resolved measurements (405-750 nm) can be recorded by each illumination fiber, and correspond to the source-detector separations mentioned above. The optimal number of wavelengths required to reconstruct images may be determined from simulated and experimental phantom data. Data acquisition can be optimized to ensure that adequate signal-to-noise ratio (SNR) is to be acquired at all wavelengths and within the dynamic range of the light detectors, i.e. the spectrometers. An automated variable-integration-time routine may be implemented, and the optimal approach to maximize SNR may be investigated across the hemoglobin-sensitive spectral range.

FIG. 18B shows a tomogram obtained using the reconstruction algorithm and a mesh specifically for the biopsy device 110, 210, 310, and 410. For instance, a mesh generator such as CGALmesh, for instance, may be used to convert coordinates of regions around the biopsy device into a tetrahedral mesh compatible with MMC. The surrounding tissue can be approximated as being a cylinder (e.g., radius=15 mm, height=30 mm) that is divided in a certain number of quadrants (equal to the number of fibers of the biopsy device 110). Each quadrant is then radially divided into subregions. Considering the case of 32 fibers, the mesh is divided in 32 quadrants, each of them then subdivided radially in 6, yielding to 192 regions around the detection/excitation area of the biopsy device in accordance with the present disclosure. The media is also divided in 32 above and under this zone. In that case there can be a total of 256 regions.

As can be understood, the examples described above and illustrated are intended to be exemplary only. The scope is indicated by the appended claims.

1. A biopsy device comprising:
a cannula body having a longitudinal axis and a probing region extending along the longitudinal axis, the probing region having a sample receiving window defined therein for receiving a sample of a surrounding tissue when performing a biopsy; and
a plurality of optical fibers mounted along an exterior portion of the cannula body, each of the plurality of optical fibers having a fiber end in the probing region of the cannula body and another fiber end adapted to be optically connectable to at least one of at least one light generator and at least one light detector, at least one of the plurality of optical fibers being adapted to illuminate the surrounding tissue with an optical signal generated by the at least one light generator and at least one of the plurality of optical fibers being adapted to detect an optical signal response with the at least one light detector, the optical signal response being caused by the propagation of the optical signal in the surrounding tissue.

2. The biopsy device of claim 1, wherein the plurality of optical fibers are circumferentially spaced from one another.

3. The biopsy device of claim 1, wherein each one of the fiber ends of the plurality of optical fibers are circumferentially aligned with one another.

4. The biopsy device of claim 3, wherein each one of the fiber ends is optically coupled to a portion of an annular redirecting surface being abutted with the circumferentially aligned fiber ends of the plurality of optical fibers, the annular redirecting surface having a normal axis forming a non-perpendicular and non-zero angle with the longitudinal axis.

5. (canceled)

6. The biopsy device of claim 1, wherein each one of the fiber ends is optically coupled to a redirecting surface being aligned along an angled axis forming a non-zero angle with the longitudinal axis of the cannula body.

7. (canceled)

8. The biopsy device of claim 1, wherein each one of the fiber ends of the plurality of optical fibers longitudinally extends to the sample receiving window.

9. The biopsy device of claim 1, wherein the plurality optical fibers are circumferentially and evenly distributed on the exterior portion of the cannula body.

10. The biopsy device of claim 1, wherein each one of the fiber ends of the plurality of optical fibers extends at least on a circumferential side of the cannula body which is opposite to the sample receiving window.

11. The biopsy device of claim 1, wherein at least one optical fiber of the plurality of optical fibers is adapted to illuminate the surrounding tissue with an optical signal and to detect an optical signal response.

12. The biopsy device of claim 1, wherein the cannula body has a depression radially recessing therefrom, the depression having a radial depth at least equal or greater than a diameter of at least one of the plurality of optical fibers, the fiber ends of the optical fibers being in the depression.

13. The biopsy device of claim 1, wherein the cannula body includes:
an inner cannula having an inner sample receiving window and having an inner diameter; and
an outer cannula having an outer sample receiving window and having an outer diameter, the outer diameter being larger than the inner diameter of the inner cannula, the outer cannula being adapted for receiving the inner cannula along a longitudinal axis thereof in such a way that the inner cannula is rotatable relative to the outer cannula at least about the longitudinal axis;
wherein the biopsy device is an open configuration when the inner sample receiving window and the outer sample receiving window are aligned with one another and the biopsy is a closed configuration when the inner sample receiving window and the outer sample receiving window are not aligned with one another.

14. The biopsy device of claim 13, wherein the exterior portion of the cannula body on which is mounted the plurality of optical fibers is an exterior portion of the outer cannula, and wherein the plurality of optical fibers have a biocompatible protective disposed thereon.

15. A spectroscopic biopsy system for providing guidance to a mechanical biopsy procedure, the system comprising:
the biopsy device of claim 1;
at least one light generator optically coupled to at least one of the plurality of optical fibers for providing the optical signal thereto;
at least one light detector optically coupled to at least one of the plurality of optical fibers for detecting the optical signal response and for generating optical signal response data associated to the optical signal response;
a computing device operatively connected to at least one light detector, adapted for receiving the optical signal response data from at least one light detector and adapted for determining an optical property of the surrounding tissue based on the optical signal response data; and
an output device operatively connected to the computing device for displaying the determined optical property which is to be used in the guidance of the mechanical biopsy procedure.

16. The spectroscopic biopsy system of claim 15, wherein at least one light generator comprises a broadband light generator for illuminating the surrounding tissue with the optical signal comprising broadband light and wherein at least one light detector is a spectrometer for detecting the optical signal response, the determined optical property being indicative of diffuse reflectance occurring in the surrounding tissue.

17. The spectroscopic biopsy system of claim 16, wherein the computing device is further adapted to determine if the surrounding tissue comprises hemoglobin when the diffuse reflectance of the optical signal response has at least one of an increased absorption and an hemoglobin spectral signature.

18. The spectroscopic biopsy system of claim 15, wherein at least one light generator comprises a fluorescence excitation generator for illuminating the surrounding tissue with the optical signal comprising fluorescence excitation light and wherein at least one light detector is a spectrometer for detecting the optical signal response, the determined optical property being indicative of fluorescence occurring in the surrounding tissue.

19. (canceled)

20. The spectroscopic biopsy system of claim 18, wherein the computing device is further adapted to determine if the surrounding tissue comprises optical markers when the fluorescence of the optical signal response has at least one of an increased intensity in the emission spectrum of the optical markers or a fluorescence signature.

21. The spectroscopic biopsy system of claim 15, wherein the at least one light generator comprises a near infrared light generator for illuminating the surrounding tissue with the optical signal comprising near infrared light and wherein the at least one light detector is a spectrometer, the optical property being of Raman scattering occurring in the surrounding tissue.

22. (canceled)

23. The spectroscopic biopsy system of claim 15,
wherein the optical coupling between at least one light generator and the at least one of a plurality of optical fiber includes a first optical multiplexer and at least one optical switch controllable to prevent transmission of the light along a corresponding one of the at least one of the plurality of optical fibers;
wherein the optical coupling between at least one light detector and the at least one of the plurality of optical fibers includes a second multiplexer and the at least one optical switch controllable to prevent transmission of the light along a corresponding one of at least one of the plurality of optical fibers;
wherein the computing device is adapted to provide the optical signal along only one of the plurality of optical fibers using the at least one optical switch, the computing device being further adapted to detect one of a plurality of partial optical signal responses from each one of the at least one of the plurality of optical fibers using the at least one optical switch, the computing device being further adapted to determine the optical property based on the plurality of partial optical signal responses.

24. The spectroscopic biopsy system of claim 23, wherein said detection of the plurality of partial optical signal responses for each one of the at least one of the plurality of optical fiber is performed sequentially in time, the computing device being further adapted to construct a tomogram representative of the optical property distribution in the surrounding tissue based on said pluralities of partial optical signal responses.

25.-28. (canceled)
